(19) United States
(12) Patent Application Publication
(10) Pub. No.: US 2023/0210781 A1
(43) Pub. Date: Jul. 6, 2023

(54) CONTROLLED RELEASE OF SELF-EMBEDDING PARTICLES FOR LOCALIZED DRUG DELIVERY

(71) Applicant: JANSSEN BIOTECH, INC., Horsham, PA (US)

(72) Inventors: Scott UHLAND, Sudbury, MA (US); Mihee KIM, San Diego, CA (US); Manish MONGA, Spring House, PA (US); Samuel SPIGELMAN, Raritan, NJ (US)

(21) Appl. No.: 18/009,135

(22) PCT Filed: Jun. 10, 2021
(86) PCT No.: PCT/IB2021/055116

§ 371 Date: Dec. 8, 2022

Related U.S. Application Data
(60) Provisional application No. 63/037,959, filed on Jun. 11, 2020.

(51) Int. Cl. A61K 9/48; A61K 31/7068; A61K 33/243; A61K 31/282; A61K 31/513; A61K 39/395

(52) U.S. Cl. CPC A61K 9/4808; A61K 9/4891; A61K 9/4833; A61K 9/4866; A61K 31/7068; A61K 33/243; A61K 31/282; A61K 31/513; A61K 39/3955

(57) ABSTRACT

A drug delivery device includes an enteric capsule enclosing an internal volume and a plurality of drug containing particles positioned within the internal volume. Each of the plurality of drug containing particles includes a matrix body and an active pharmaceutical ingredient (API) distributed within the matrix body. The plurality of drug containing particles are configured to penetrate tissue, such as intestinal mucosa.

### BACKGROUND

Drug delivery refers to the administration of a pharmaceutical compound (including large and small molecule pharmaceuticals, hereinafter “drug” or “drugs”) to achieve a therapeutic effect in humans and animals. A variety of drug delivery routes have been developed and include intravenous, intramuscular, intranasal, intradermal, and oral administration, amongst others. The mechanism by which a drug is absorbed, as well as the nature of the drug, are significant factors that determine which delivery route is appropriate for achieving highest bioactivity and effectivity of a drug.

Orally administered drugs are generally transported and absorbed in the gastrointestinal (GI) tract, which includes the upper GI tract (e.g., mouth, esophagus, stomach, and the initial portion of the small intestine) and the lower GI tract (the remainder of the small intestine, the large intestine, and rectum). For some diseases that occur along the GI tract, an orally bioavailable may be required. The local application of a compound along the GI tract is analogous to topical creams that can treat localized inflammation on the skin. Examples of maladies that might benefit this locally administered approach are Irritable Bowel Syndrome (IBS) and Inflammatory Bowel Diseases (IBDs), such as Crohn's disease, and ulcerative colitis.

Even under diseased conditions, the GI tract may exhibit a variety of barriers that can inhibit delivery of therapeutic levels of an orally administered drug at the disease site. Examples of such barriers can include one or more of the following: acidic and enzymatic degradation within the stomach, pH variations along the small intestine between individuals, microflora, e.g. intestinal bacteria in the colon that degrade the drug, non-organic intestinal contents, altered epithelial function that affects drug absorption, gastric contents and emptying/retention time. Furthermore, dissolution can be a challenge for water soluble drugs, as less water in the colon and viscosity of colonic luminal contents progressively increase as a drug transits from the ascending colon towards the descending colon. Absorption enhancers (e.g., non-steroidal anti-inflammatory drugs (NSAIDs), surfactants, fatty acids, etc.) can be required to overcome these barriers.

Accordingly, there remains a need for improved systems and methods that address these challenges and provide improved localized delivery of drugs.

### SUMMARY

In an embodiment, a drug delivery device is provided. The drug delivery device can include an enteric capsule and a plurality of drug containing particles. The enteric capsule can enclose an internal volume. The plurality of drug containing particles can be positioned within the internal volume. Each of the plurality of drug containing particles can further include a matrix body and an active pharmaceutical ingredient (API) distributed within the matrix body. The plurality of drug containing particles can be configured to penetrate tissue.

In another embodiment, the enteric capsule includes an outer layer overlying an inner layer. The outer layer can be soluble within the stomach and the inner layer can be soluble within the small or large intestine.

In another embodiment, the enteric capsule can include a single outer layer.

In another embodiment, the API can be selected from one or more of peptides, antisense oligonucleotides greater than 500 Da, cytokines, monoclonal antibodies, chemotherapy drugs, PD-1 inhibitors, PD-L1 inhibitors, and combinations thereof.

The chemotherapy drugs can be selected from one or more of Gemcitabine, Cisplatin, Carboplatin, Fluorouracil (5FU), and combinations thereof.

The PD-1 inhibitors can be selected from one or more of Pembrolizumab, Nivolumab, Cemiplimab, and combinations thereof.

The PD-L1 inhibitors can be selected from one or more of Atezolizunab, Avelumab, Durvalumab, and combinations thereof.

In another embodiment, the matrix body can be formed from biodegradable polymers.

The biodegradable polymers can be selected from one or more of poly(lactic-co-glycolic acid) [PLGA] polymers, PLGA copolymers, poly(caprolactone)s (PCLs), poly(alkyl cyanoacrylates) (PACAs), poly(ortho esters), poly(anhydrides), poly(amides), poly(ester amides), poly(phosphoesters), microbial release polymers, and combinations thereof.

In another embodiment, the PLGA polymer can be poly(glycolic acid) (PGA) or poly(D,L-lactic acid) (PLA).

In another embodiment, the PLGA copolymer can be a copolymer of poly(D,L-lactic-co-glycolic acid) [PLGA] or a copolymer of polyester and polyethylene glycol (PEG).

In another embodiment, an aspect ratio of the plurality of drug containing particles can be within the range from about 5 to about 100.

In another embodiment, the plurality of drug containing particles can have a shape including at least one vertex.

In another embodiment, the plurality of drug containing particles can have an elastic modulus within the range from about 1 GPa to about 10 GPa.

In another embodiment, an axial failure force of the plurality of drug containing particles can be within the range from about 1 N to about 10 N.

In another embodiment, the plurality of drug containing particles can have a sharpness within the range from about 0.1 μm to about 20 μm.

In another embodiment, the surface of at least a portion of the drug containing particles can be functionalized with a mucoadhesive.

In an embodiment, a method of preparing a drug delivery composition is provided. The method can include forming a plurality of drug containing particles. Each of the plurality of drug containing particles can include a matrix body and an active pharmaceutical ingredient (API) distributed within the matrix body. The drug containing particles can be further configured to penetrate tissue. The method can also include enclosing the plurality of drug containing particles in an internal volume of an enteric capsule. The enteric capsule can be configured to release the plurality of drug containing particles from the cavity after placement within a gastrointestinal tract of a patient for a predetermined amount of time.

In another embodiment, the enteric capsule can include an outer layer overlying an inner layer. The outer layer can be soluble within the stomach and the inner layer can be soluble within the small or large intestine.

In another embodiment, the enteric capsule includes a single outer layer.

In another embodiment, the API can be selected from one or more of peptides, antisense oligonucleotides greater than 500 Da, cytokines, monoclonal antibodies, chemotherapy drugs, PD-1 inhibitors, PD-L1 inhibitors, and combinations thereof.

In another embodiment, the chemotherapy drugs can be selected from one or more of Gemcitabine, Cisplatin, Carboplatin, Fluorouracil (5FU), and combinations thereof.

In another embodiment, the PD-1 inhibitors can be selected from one or more of Pembrolizumab, Nivolumab, Cemiplimab, and combinations thereof.

In another embodiment, the PD-L1 inhibitors can be selected from one or more of Atezolizumab, Avelumab, Durvalumab, and combinations thereof.

In another embodiment, the matrix body can be formed from a biodegradable polymer.

The biodegradable polymer can be selected from one or more of poly(lactic-co-glycolic acid) [PLGA] polymers, PLGA copolymers, poly(caprolactone)s (PCLs), poly(alkyl cyanoacrylates) (PACAs), poly(ortho esters), poly(anhydrides), poly(amides), poly(ester amides), poly(phosphoesters), microbial release polymers, and combinations thereof.

In another embodiment, the PLGA polymer can be poly(glycolic acid) (PGA) or poly(D,L-lactic acid) (PLA).

In another embodiment, the PLGA copolymer can be a copolymer of poly(D,L-lactic-co-glycolic acid) [PLGA] or a copolymer of polyester and polyethylene glycol (PEG).

In another embodiment, an aspect ratio of the plurality of particles can be within the range from about 5 to about 100

In another embodiment, the plurality of drug containing particles can have a shape including at least one vertex.

In another embodiment, the plurality of drug containing particles can have an elastic modulus within the range from about 1 GPa to about 10 GPa.

In another embodiment, an axial failure force of the plurality of drug containing particles can be within the range from about 1 N to about 10 N.

In another embodiment, the plurality of drug containing particles can have a sharpness within the range from about 0.1 μm to about 20 μm.

In another embodiment, the surface of at least a portion of the drug containing particles can be functionalized with a mucoadhesive.

In another embodiment, forming the plurality of drug containing particles can include casting a liquid precursor of the drug containing particles, solidifying the liquid precursor to form a sheet of the drug containing particles, urging a portion of the sheet within cavities of a mold to form discrete drug containing particles, and removing the discrete drug containing particles from the mold.

In an embodiment, a method of orally delivering a drug to the gastrointestinal tract is provided and includes orally administering an effective amount a drug delivery device. The drug delivery device can include an enteric capsule and a plurality of drug containing particles. The enteric capsule can enclose an internal volume. The plurality of drug containing particles can be positioned within the internal volume. Each of the plurality of drug containing particles can further include a matrix body and an active pharmaceutical ingredient (API) distributed within the matrix body. The plurality of drug containing particles can be configured to penetrate tissue.

In another embodiment, the method further includes orally administering one or more second capsules that are substantially insoluble within the GI tract.

In another embodiment, the surface of the drug containing particles can be functionalized with a compound configured to promote adsorption of the drug containing particles to the one or more second capsules.

In another embodiment, the one or more second capsules can be configured to swell within the GI tract.

In another embodiment, the dimensions of the second capsules can be independently selected within the range from about 5 mm to about 25 mm.

In another embodiment, the second capsules can be configured for electrostatic affinity with the drug containing particles.

### BRIEF DESCRIPTION OF THE DRAWINGS

Embodiments of the disclosure can be more fully understood from the following detailed description taken in conjunction with the accompanying drawings, in which:

FIG. 1 is a diagram of a patient illustrating one exemplary embodiment of an orally administered drug delivery device traveling through the gastrointestinal (GI) tract;

FIG. 2A is a diagram illustrating one exemplary embodiment of the drug delivery device of FIG. 1 including a plurality of drug containing particles housed within a capsule including an outer coating layer and an inner coating layer;

FIG. 2B is a diagram illustrating the drug delivery device of FIG. 2A after dissolution of the outer coating layer;

FIG. 2C is a schematic diagram illustrating the drug delivery device of FIG. 2A after dissolution of the outer coating layer and partial dissolution of the inner coating layer;

FIG. 2D is a schematic diagram illustrating release of the drug containing particles from the drug delivery device of FIG. 2A after dissolution of the outer coating layer and the inner coating layer;

FIG. 3A is a diagram illustrating another exemplary embodiment of the drug delivery device of FIG. 1 including a plurality of drug containing particles housed within a capsule including a single, outer coating layer;

FIG. 3B is a schematic diagram illustrating release of the drug containing particles from the drug delivery device of FIG. 3A after dissolution of the outer coating layer;

FIG. 4 is a schematic diagram illustrating a composite microstructure of the drug containing particles of FIG. 1;

FIGS. 5A, 5B, and 5C illustrate one exemplary embodiment of a method of manufacturing the drug containing particles; (5A) a composition of the drug containing particles is cast as a sheet and solidified; (5B) the sheet and a mold containing a plurality of cavities is compressed between rollers to fill the cavities with the drug containing particle composition; (5C) the drug containing particles are released from the mold cavities;

FIG. 6 is a diagram illustrating interaction (e.g., contact and/or penetration) of the drug containing particles of the drug delivery device of FIG. 1 with intestinal mucosa;

FIG. 7A is a micrograph illustrating one exemplary embodiment of a shape of the drug containing particles of the drug delivery device of FIG. 1 having a barbed shape;

FIG. 7B is a micrograph illustrating another exemplary embodiment of a shape of the drug containing particles of the drug delivery device of FIG. 1 having a conical shape;

FIG. 7C is a micrograph illustrating another exemplary embodiment of a shape of the drug containing particles of the drug delivery device of FIG. 1 having a cylindrical shape;

FIG. 7D is a micrograph illustrating another exemplary embodiment of a shape of the drug containing particles of the drug delivery device of FIG. 1 having a rectangular shape;

FIG. 7E is a micrograph illustrating another exemplary embodiment of a shape of the drug containing particles of the drug delivery device of FIG. 1 having a star-like shape;

FIG. 8A is a diagram illustrating an embodiment of the drug containing particles of the drug delivery device of FIG. 1 including self-orienting arms approaching microvilli extending from intestinal side walls;

FIG. 8B is a diagram illustrating the drug containing particles of FIG. 8A engaging the microvilli;

FIG. 9 is a diagram illustrating use of a second capsule administered in tandem with the drug delivery device of FIG. 1 to facilitate interaction of the drug containing particles with intestinal mucosa; and

FIG. 10 is a diagram illustrating interaction (e.g., contact and/or penetration) of the drug containing particles of the drug delivery device with tissues of the bladder.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment can be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

A variety of “pill-like” devices have been developed to deliver therapeutics to the intestinal wall. However, these pill-like devices possess deficiencies. In one aspect, existing pill-like devices can be limited, either due to complexity of manufacture (e.g., they include subsystems with moving parts) or manufacturing cost. Thus, their reliability is questionable. In another aspect, existing pill-like devices can be limited in the dose of a drug solution that can be delivered to a specific location within the intestinal tract. This limitation in the drug pay-load result from the structure of the device, where a majority of the volume of the device is occupied by the actuation mechanism and a relatively small volume is occupied by the therapeutic. In a further aspect, another limitation to these devices is their limited area of treatment. Existing pill-like devices can engage with a very limited region of the tissue. As a result, the likelihood that the device locally engages with the diseased tissue area, as compared to engaging undiseased tissue area, is small, preventing the device from achieving the desired therapeutic benefit from true local administration.

In general, embodiments of the present disclosure provide improved drug delivery systems and methods for using the same. A drug delivery system can include drug containing particles housed within an enteric capsule. Upon dissolution of the enteric capsule, the drug containing particles are released into the GI tract. The drug containing particles possess properties that enable them to adsorb into and/or embed within the lining of the GI tract (e.g., mucosa). Examples of such properties include physical properties (e.g., size, shape), mechanical properties (e.g., modulus), and/or chemical properties (e.g., surface functionality, composition).

The drug delivery system can optionally include particles that are relatively insoluble within the GI tract, as compared to the enteric capsule. These insoluble particles can physically urge the drug containing particles towards the mucosa, facilitating initial penetration of drug containing particles within the mucosa and/or further penetration of drug containing particles that have already penetrated the mucosa.

As discussed in detail below, the disclosed systems and methods address limitations of existing drug delivery systems, providing higher drug payload throughout the intestine. These systems and methods further provide a pathway to overcome general challenges to local delivery of drugs to the intestinal mucosa.

Embodiments of the drug delivery system are discussed in the context of oral administration and drug absorption by the GI tract. It can be understood, however, that further embodiments of the drug delivery system can be administered by other routes for absorption by other tissue without limit. Examples of other tissues that can be treated include, but are not limited to, the bladder and reproductive tract.

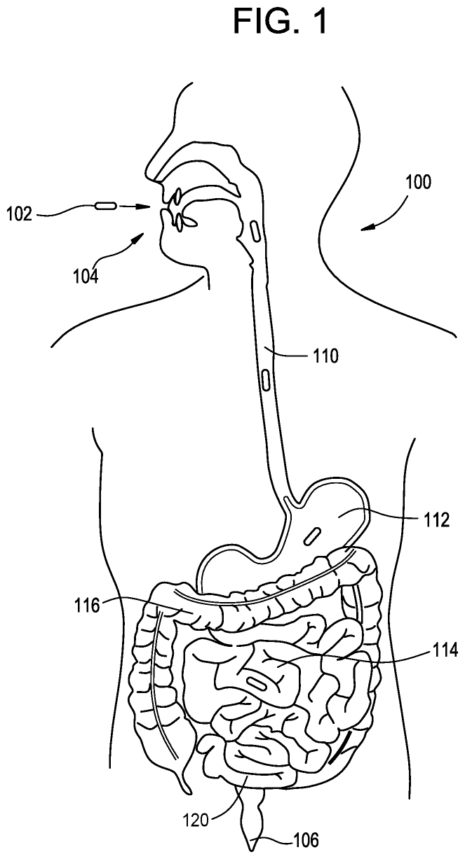

FIG. 1 is a diagram of a patient 100 illustrating one exemplary embodiment of an orally administered drug delivery device 102 traveling through the GI tract. The GI tract is a series of hollow organs forming a long, twisting tube that extends from the mouth 104 to the anus 106. These hollow organs include the esophagus 110, the stomach 112, the small intestine 114, the large intestine 116, the rectum 120, and the anus 106. The drug delivery device 102 is received within the mouth 104 and passes through the GI tract to a desired treatment area.

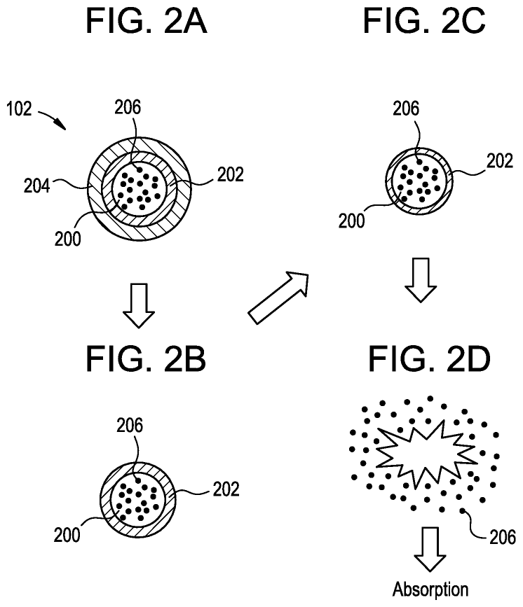

FIG. 2A illustrates one exemplary embodiment of the drug delivery device 102 in greater detail. As shown, the drug delivery device 102 is in the form of a capsule that defines an internal volume or cavity 200. The capsule includes an inner layer 202 and an outer layer 204 overlying the inner layer 202, where an interior facing wall of the inner layer bounds the cavity 200. A plurality of drug containing particles 206 are disposed within the cavity 200.

The outer layer 204 can be an enteric coating configured to selectively dissolve within the GI tract. As an example, the outer layer 204 can be configured to resist dissolution within the more strongly acidic conditions of the stomach and readily dissolve within portions of the GI tract with a higher pH, for example the small intestine 114 (FIG. 2B). By way of example, dissolution can begin at a pH of about 4 or higher, at a pH of about 5 or higher, at a pH of about 6 or higher, or at a pH of about 7 or higher.

The inner layer 202 can be further configured to dissolve within the small intestine 114 and/or large intestine 116 at a controlled rate (FIG. 2C). In this manner, the capsule prevents the drug containing particles 206 from being exposed in the stomach 112 and delays release in the intestines 114, 116. The initiation of release, where the inner layer 202 dissolves to the point where the cavity 200 is no longer completely enclosed by the inner layer 202 (FIG. 2D), and the rate of dissolution is controlled by either exposure to higher pH and/or exposure to microflora/bacteria in the small and/or large intestine 114, 116. The inner layer 202 can be additionally configured to provide one or more of the following: manufacturability, dispersion of the drug containing particles 206, cohesiveness of the drug containing particles 206, and osmotic dissolution of the drug containing particles 206.

The outer layer 204 and the inner layer 202 can be formed from gastric resistant compounds. In general, there are a variety of polymers that can be used to achieve controlled gastric resistance. Examples include fatty acids, waxes, shellac, plastics, and plant fibers. In certain embodiments, the outer layer can be formed from any one of cellulose acetate phthalate, cellulose acetate trimellitate, polyvinyl acetate phthalate, hydroxypropyl methylcellulose phthalate, Hydroxypropyl methylcellulose acetate succinate, cellulose, copolymers of methacrylic acid and ethyl acrylate (1:1 ratio), or copolymers of methacrylic acid and methylmethacrylate (1:1 or 1:2 ratio). In further embodiments, the inner layer can be formed from magnesium stearate, stearic acid, gelatin, microcrystalline cellulose powder, glycerin, citric acid, polyethylene glycol, or Hydroxypropyl methylcellulose.

In further embodiments, the outer layer 204 and/or the inner layer 202 can be independently selected from materials having functionality discussed in “Degradable Controlled-Release Polymers and Polymeric Nanoparticles: Mechanisms of Controlling Drug Release,” Chem. Rev., 116(4), 2602-2663 (2016), the entirety of which is incorporated by reference.

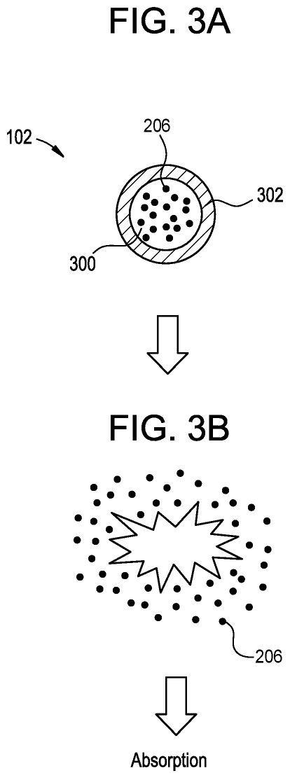

FIG. 3A illustrates an alternative embodiment of the drug delivery device 102. The drug delivery device 102 of FIG. 3A is similar to that of FIG. 2A, except that the inner layer 202 is omitted. That is, the drug delivery device of FIG. 3A includes a capsule that defines an internal volume or cavity 300 with a single layer 302 (e.g., the outer layer). As discussed above, the outer layer 302 can be an enteric coating configured to resist dissolution within the acidic conditions of the stomach and dissolve within the alkaline pH of the large/lower intestine. The initiation of release occurs when the outer layer 302 dissolves to the point where the cavity 300 is no longer completely enclosed (FIG. 3B).

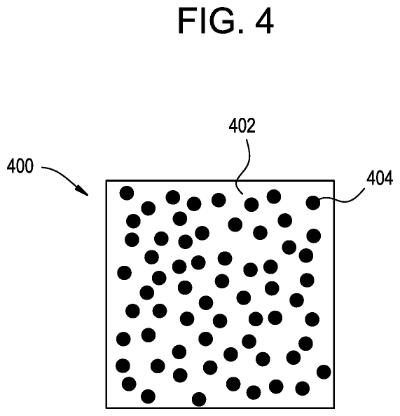

In certain embodiments, the drug containing particles 206 possess a composite microstructure. As shown in FIG. 4, the composite 400 includes a matrix body 402 and an active pharmaceutical ingredient (API) 404 embedded within the matrix body 402. The distribution of API particles within the matrix body 402 can be substantially homogeneous or non-homogenous. The volumetric fractions of the API 404 within the matrix body 402 can range from about 0 to about 0.75. Alternatively, the volumetric fractions of the API 404 within the matrix body 402 can range from about 0.1 to about 0.3, from about 0.1 to about 0.4, from about 0.1 to about 0.5, from about 0.1 to about 0.6, from about 0.2 to about 0.3, from about 0.2 to about 0.4, from about 0.2 to about 0.5, or from about 0.2 to about 0.6. The API 404 may be in a solution phase with a solvent within the matrix body 402, partially soluble within the matrix body 402, or a solid suspended within the matrix body 402. For example, certain embodiments of the drug containing particles 206 can have the API 404 in a nano- or microparticle form, suspended within an excipient matrix body where the API 404 is about 50 vol. % of the matrix body volume.

Embodiments of matrix body 402 can be formed from a variety of different materials. Examples can include biodegradable polymers. The biodegradable polymers can include, but are not limited to, poly(lactic-co-glycolic acid) or PLGA polymers, PLGA copolymers, poly(caprolactone)s (PCLs), poly(alkyl cyanoacrylates) (PACAs), poly(ortho esters), poly(anhydrides), poly(amides), poly(ester amides), poly(phosphoesters), or microbial release polymers. Examples of PLGA polymers can include poly(glycolic acid) (PGA) or poly(D,L-lactic acid) (PLA). Examples of PLGA copolymers can include poly(D,L-lactic-co-glycolic acid) (PLGA) or polyester-polyethylene glycol (PEG) copolymers. Further embodiments of excipient materials that can form the matrix body are discussed in “Excipient selection for compounded pharmaceutical capsules: they're only fillers, right?” Australian Journal of Pharmacy, 98(1164), 78-83 (2017), the entirety of which is incorporated by reference.

Embodiments of API 404 can be formed from a variety of different compounds. Examples can include:
Small molecules that need a longer retention time for better bioavailability or absorptions.
Small molecules such as peptides and antisense oligonucleotides that are greater than 500 Da and therefore have poor properties to readily pass through the epithelial barrier (e.g., proteins, such as cytokines or signaling molecules, and monoclonal antibodies.)
Drugs for bladder cancer, such as chemotherapy drugs suitable for treatment of various neoplasms. Such chemotherapy drugs can include, but are not limited to, Gemcitabine, Cisplatin, Carboplatin, Fluorouracil (5FU), PD-1 inhibitors, PD-L1 inhibitors. PD-1 inhibitors can include, but are not limited to, Pembrolizumab, Nivolumab, Cemiplimab. PD-L1 inhibitors can include, but are not limited to, Atezolizumab, Avelumab, Durvalumab.

Non-limiting examples of other APIs 404 include cisplatin or carboplatin), paclitaxel, docetaxel, TIP (paclitaxel/Taxol, ifosfamide, and cisplatin/Platinol), VeIP (vinblastine, ifosfamide, and cisplatin/Platinol), VIP (etoposide/VP-16, ifosfamide, and cisplatin/Platinol), VAC (vincristine, dactinomycin, and cyclophosphamide), Albumin bound paclitaxel, Altretamine, Capecitabine, Cyclophosphamide, Etoposide, Gemcitabine, Ifosfamide, Irinotecan, Liposomal doxorubicin, Melphalan, Pemetrexed, Topotecan, Vinorelbine, and combinations thereof.

In certain embodiments, the drug containing particles 206 can be formed by a molding process. The molding process is discussed in detail within one or more of the following, the entirety of each of which is incorporated by reference.
U.S. Pat. No. 7,976,759, entitled “System and Method For Producing Particles and Patterned Films”
“Shape-specific, Mono-disperse Nano-molding of Protein Particles,” Kelly, J. Y.; DeSimone*, J. M. J. Am. Chem. Soc. 2008, 130, 5438-5439.
“Microfabricated Particles for Engineered Drug Therapies: Elucidation into the Mechanisms of Cellular Internalization of PRINT® Particles,” Gratton, S. E. A.; Napier, M. E.; Ropp, P. A.; Tian, S. DeSimone*, J. M.; Pharm. Res. 2008, 25, 2845-2852.
“Reductively Labile PRINT® Nanoparticles for the Delivery of Doxorubicin to HeLa Cells,” Petros, R. A.; Ropp, P. A.; DeSimone*, J. M.; J. Am. Chem. Soc. 2008, 130, 5008-5009.
“The Pursuit of a Scalable Nano-fabrication Platform for Use in Material and Life Science Applications,” Gratton, S. E. A.; Williams, S. S.; Napier, M. E.; Pohlhaus, P. D.; Zhou, Z.; Wiles, K. B.; Maynor, B. B.; Shen, C.; Olafsen, T.; Samulski, E. T.; DeSimone*, J. M. Accounts of Chemical Research 2008, 41, 1685-1695.
“Nanoparticle Drug Delivery Platform,” Napier, M. E.; DeSimone*; J. M. Polymer Reviews 2007, 47, 321-327.
“Nanofabricated Particles for Engineered Drug Therapies: A Preliminary Biodistribution Study of PRINT® Nanoparticles,” Gratton, S. E. A.; Pohlhaus, P. D.; Lee, J.; Guo, J.; Cho, M. J.; DeSimone*, J. M. J. Controlled Release 2007, 121, 10-18.
“Imparting Size, Shape, and Composition Control of Materials for Nanomedicine;” Eulis, L.; DuPont, J.; DeSimone*, J. M. Chem. Soc. Rev. 2006, 35, 1095-1104.
“Direct Fabrication and Harvesting of Monodisperse, Shape Specific Nano-Biomaterials,” Rolland, J. P.; Maynor, B. W.; Euliss, L. E.; Exner, A. E.; Denison, G. M.; DeSimone*, J. M J. Am. Chem. Soc. 2005, 127, 10096-10100.

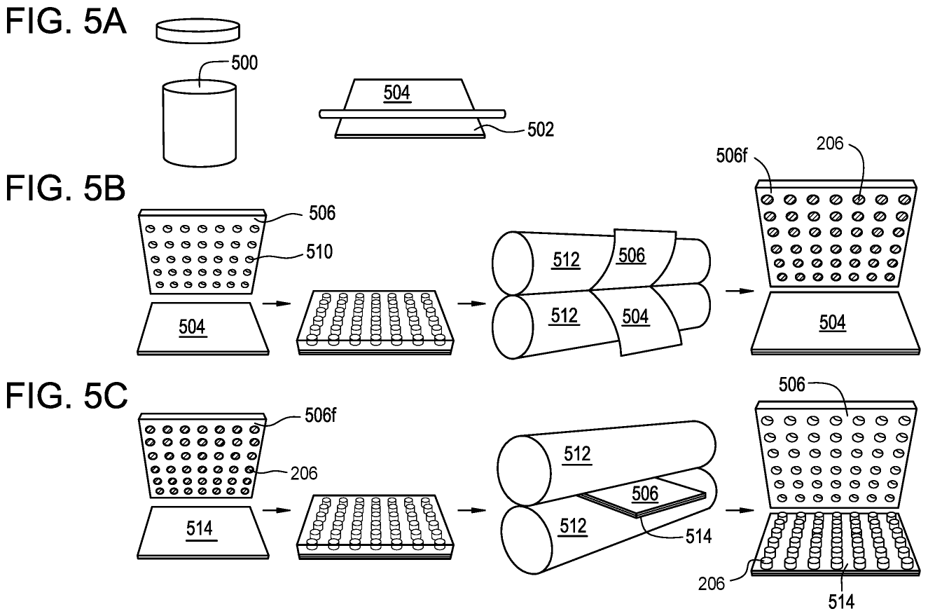

A brief discussion of the molding process is provided below with reference to FIGS. 5A-5C. As shown in FIG. 5A, a liquid precursor 500 of the drug containing particles 206 is cast on a substrate 502. Solvent is removed under heat to generate a solid state solution film having the composite microstructure, also referred to as a delivery sheet 504. As shown in FIG. 5B, a mold 506 including a plurality of mold cavities 510 having a predetermined geometry is brought into contact with the delivery sheet 504. The mold 506 and delivery sheet 504 are passed through heated rollers 512 and split. High pressure and heat between the rollers 512 softens the delivery sheet 504, causing a portion of the delivery sheet 504 to flow and fill the mold cavities 510 (filled mold 506f), forming discrete drug containing particles 206. Subsequently, the discrete drug delivery particles 206 can be removed from the mold 506. In one embodiment, as shown in FIG. 5C, the filled mold 506f is brought into contact with a high energy film or excipient layer 514 and passed through the heated rollers 512 without splitting. After cooling, the mold 506 is removed to reveal an array of discrete drug containing particles 206 on the high energy film or excipient layer 514. In another embodiment, the discrete drug containing particles 206 can be directly removed from the mold 506 after the operations of FIG. 5B. In either case, the drug containing particles 206 mimic the size and shape of the mold cavities 510.

In alternative embodiments, the film casting operation of FIG. 5A can be omitted. Instead, the liquid precursor 500 can be applied directly to the mold 506. Nipping the mold 506 between a roller 512 and a surface causes the liquid precursor 500 to enter the mold cavities 510. Once the liquid precursor 500 fills the mold cavities 510, solvent can be removed to solidify the liquid precursor 500.

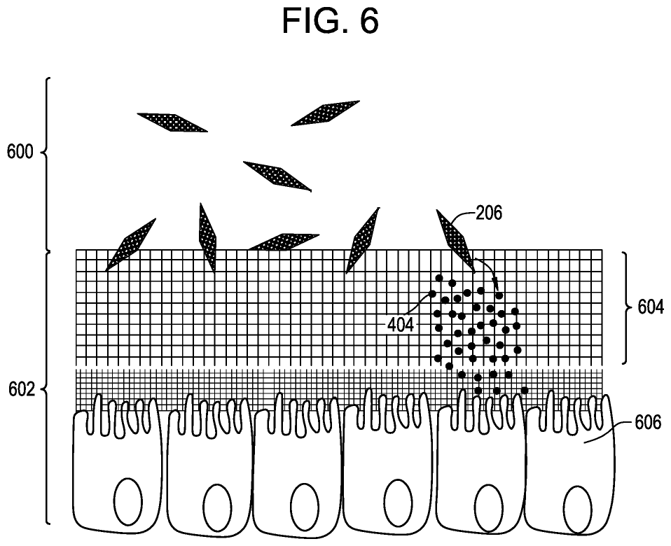

As illustrated in FIG. 6, the drug containing particles 206 are configured to travel through the GI tract lumen 600 and interact with (e.g., contact and/or at least partially embed within) the GI tract wall 602, such as mucosal lining 604 of the intestines 114, 116 once released from the drug delivery device 102. Because the matrix body 402 is biodegradable, the API 404 within the drug containing particles 206 is released as the matrix body 402 decomposes. Interaction of the drug containing particles 206 with the mucosa 604 provides a relatively long residence time for the released API 404 to move through the mucosa 604 and reach the enterocytes 606 that function as intestinal absorptive cells.

The drug containing particles 206 can possess one or more physical properties, mechanical properties, or chemical properties, in any combination, that facilitate engagement of the drug containing particles 206 with the intestinal mucosa 604. Beneficially, the above-discussed molding process can provide independent control of these properties with a high degree of precision and reproducibility.

Examples of physical properties can include size and shape. The size of the drug containing particles 206 can be configured to facilitate engagement with the mucosa 604 by engaging with microvilli, which have a length of about 1 μm. The size of the drug containing particles 206 can also be configured to reach the depths of the target epithelium and deep mucosa, which are up to about 200 μm to 500 μm. Accordingly, non-limiting embodiments, the dimensions of the drug containing particles 206 can be independently selected from the range of about 5 μm to about 500 μm.

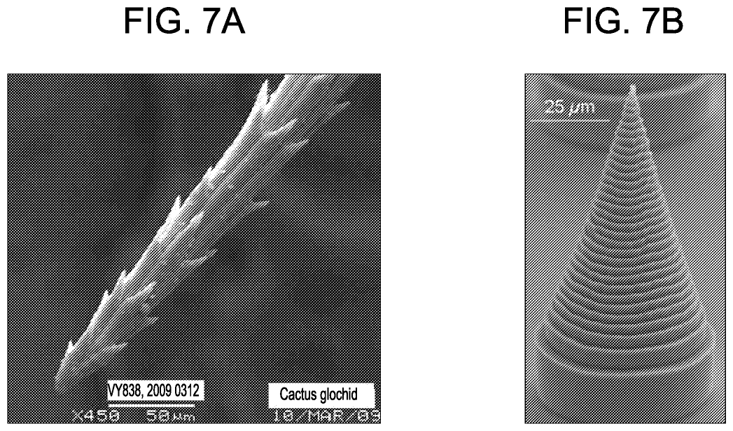

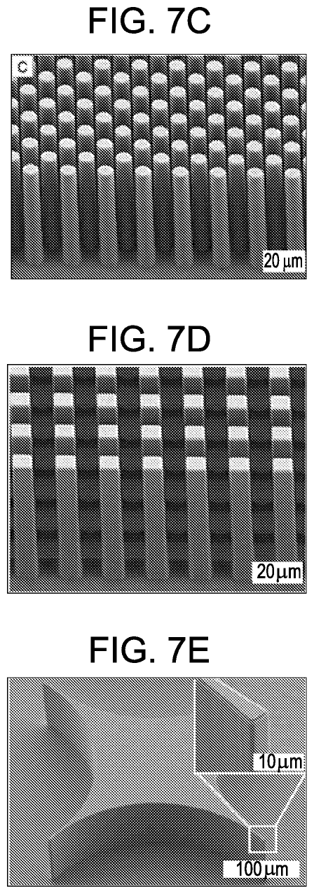

The shape of the drug containing particles 206 can also facilitate engagement with the mucosa 604 by providing a high aspect ratio (length:diameter) or “needle-like” geometry (e.g., similar to cactus needles or porcupine quills). In general, the shape of the drug containing particles 206 can adopt the form of any geometry that includes surface features that provide stress concentrators to augment penetration. Examples of such geometries can include cones, pyramids with either smooth or stepped surfaces, star-like shapes with smooth or textured surfaces, or a combination of simple geometries, such as rods or rectangles. Examples are illustrated in FIGS. 7A-7B. As shown in the micrograph of FIG. 7A, the particle shape is elongated and can include rearward facing barbs. The barbs can concentrate stress on smaller points, instead of the entire surface area of the body. As a result, lower pressure is applied by numerous barbs of small area to accomplish the same result as large pressure on one area. FIG. 7B is a micrograph of another embodiment of the drug containing particles having a conical shape with annular, surface features. Examples of cylindrical, rectangular, and star-shapes are further illustrated in the micrographs of FIGS. 7C-7E, respectively.

Such shapes can provide the matrix body 402 with a sharpness sufficient to penetrate the mucosa 604. In general, sharpness can represent the amount of force required to penetrate a standard membrane. Thus, higher sharpness facilitates penetration. In non-limiting embodiments, the aspect ratio of the matrix body 402 can be selected from the range of about 5 to about 100. In further non-limiting embodiments, the sharpness of the matrix body 402 can be selected from the range of about 0.1 μm to about 20 μm.

Without being bound by theory, it is expected that sharp particles penetrate the mucosa 604 by one or more of the following mechanisms, alone or in combination. In one aspect, penetration can occur through natural dispersion into the local fluids that are present or in contact within the GI mucosa 604. In another aspect, penetration can occur due to locomotion of the intestines as the muscles contract and expand.

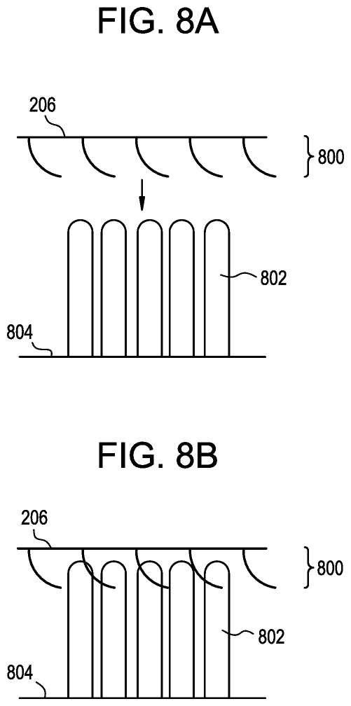

In further embodiments, the drug containing particles 206 can include self-orienting side appendages/arms 800 to control orientation. In general, the orientation of the drug-containing particles 206 can be such that the penetrating features (e.g., barbs) are positioned adjacent to the surface of the tissue to be penetrated. In certain embodiments, the orientation can position a longitudinal axis of at least a portion of the drug-containing particles 206 at an angle of approximately between about 45 degrees to −45 degrees with respect to the tissue surface. In one example, shown in FIG. 8A, the microvilli 802 extend out from the surface of the intestinal side walls 804, while the arms 800 extend out from the surface of the drug containing particles 206. The configuration of the arms 800 can adopt a variety of shapes, including straight, curved, barbed, conic, and combinations thereof. So configured, as shown in FIG. 8B, the arms 800 can physically engage the microvilli 802.

Examples of mechanical properties can include modulus (e.g., elastic modulus). In general, elastic modulus is a material property characterizing the resistance of a material to elastic deformation. In order to avoid substantial elastic deformation when interacting with the mucosa, it is desirable for the matrix body 402 and/or arms 800 of the drug containing particles 206 to possess an elastic modulus greater than that of the mucosa 604. In non-limiting embodiments, the elastic modulus of the drug containing particles 206 can be from the range of about 1 GPa to about 10 GPa (e.g., about 3 GPa to about 6 GPa).

Further examples of mechanical properties can include mechanical strength. In general, the mechanical strength of the drug containing particles 206 (e.g., the matrix body 402 and/or arms 800) can be sufficient to inhibit failure (e.g., fracture) during engagement with the intestinal sidewall 804. In non-limiting embodiments, an axial failure force of the drug containing particles 206, in compression or tension, can be greater than 1 N (e.g., within the range from about 1 N to about 10 N).

Examples of chemical properties can include surface functionality and composition. In general, surfaces can be functionalized using a variety of compounds in order to tailor interaction between the drug containing particles 206 and mucosa 604. As an example, the surface of the matrix body 402 can be functionalized for adhesion to the mucosa 604 (e.g., mucoadhesives). Functionalizing compounds can include compounds that improve paracellular uptake by temporarily disrupting tight junctions (e.g., surfactants such as C10).

In other embodiments, functionality can include the integration of mucoadhesive chemistries onto/into the matrix of the drug containing particles 206. In general, mucoadhesion can include any bond formed between two surfaces. In the context of the present disclosure, mucoadhesion can be provide between the drug containing particle 206 and the gastro-intestinal surface. Bond(s) formed by mucoadhesion can be configured to lengthen the time of direct contact between the two surfaces. This can provide sufficient time (e.g., up to about 12 hours) for tissue penetration by the drug containing particles 206 and drug release. Embodiments of mucoadhesives can include mucoadhesive polymers such as hydrophilic polymers and/or hydrogels.

Adhesive hydrophilic polymers can include those containing carboxylic groups Examples can include, but are not limited to, polyvinyl pyrrolidone (PVP), methyl cellulose (MC), sodium carboxy-methylcellulose (SCMC), hydroxyl-propyl cellulose (HPC), and other cellulose derivatives. Examples of hydrogels can include, but are not limited to, anionic-based gels such as carbopol, polyacrylates, and cross-linked modified polyacrylates, cationic-based gels such as chitosan and derivatives, and neutral-based gels such as eudragit-NE30D.

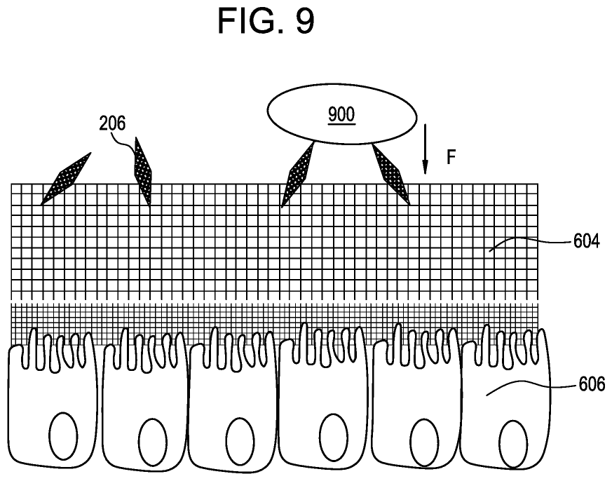

According to embodiments shown in FIG. 9, a plurality of second capsules 900 can optionally be administered in tandem with the drug delivery device 102. The second capsules 900 are substantially insoluble within the GI tract and can be configured to assist interaction of the drug containing particles 206 with the mucosa 604 by moving through the GI tract and amplifying the forces F acting on drug containing particles 206 released therein (FIG. 9).

As an example, the second capsule 900 can provide mechanical assistance. In general, the second capsule 900 can be dimensioned by a predetermined amount less than the passageway in which it travels. In this manner, the second capsule 900 can facilitate contact between the second capsules 900 and the drug containing particles 206 and be effective to push the drug containing particles 206 into the mucosa 604. In one aspect, the second capsule 900 can be relatively rigid (e.g., having an elastic modulus greater than that of the drug containing particles 206 or tissue within the GI tract). In another aspect, the second capsule 900 can be configured to swell (e.g., due to absorption of moisture from the local environment) in order to push the particles into the mucosa 604. The dimensions of the second capsule 900, in either the rigid configuration or the swelled configuration, can be independently selected from about 5 mm to about 25 mm.

As another example, the second capsule 900 can provide chemical assistance. In one aspect, chemical assistance can take the form of adsorption of the drug containing particles 206 to the mucosa 604. In another aspect, the second capsule 900 can exhibit affinity (e.g., electrostatic) with the drug containing particles 206. Similar to the mechanisms discussed above with regards to mucoadhesives, such as electrostatic and/or mechanical entanglement of polymer chains can promote temporary bonding between surfaces of the second capsule 900 and the tissue, providing mechanical coupling of the second capsule 900 with the tissue side wall. This interaction can apply greater forces on the drug containing particles 206 that are intimately in contact with the tissue surface. This applied force can further augment the penetration of the drug containing particle 206 into the target tissue for drug delivery. These interaction forces can be further enhanced by the natural muscular locomotion of the gastro-intestinal tract.

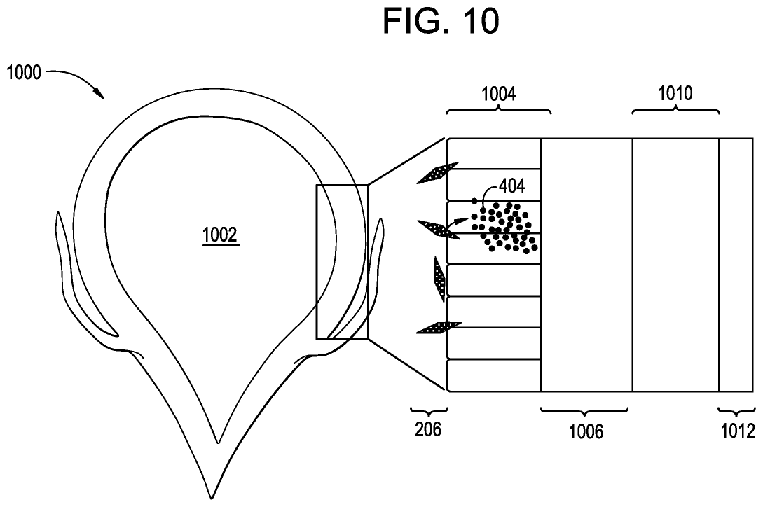

Embodiments of the drug-delivery device 102 have been discussed above in the context of oral delivery and release within the gastro-intestinal tract. However, use of the drug-delivery device 102 for delivery and release to other areas of the body are contemplated. As an example, FIG. 10 is a diagram illustrating interaction (e.g., contact and/or penetration) of the drug containing particles 206 of the drug delivery device 102 with tissues of the bladder 1000. The tissue of the bladder walls surrounds a bladder lumen 1002 includes the urothelium 1004, the lamina propria 1006, the muscularis mucosa 1010, and the adipose layer 1012. The urothelium 1004 is the innermost layer of the bladder 1000, which has a thickness of about 1 mm to about 3 mm. The lamina propria 1006 is the layer between the urothelium 1004 and muscularis mucoasa 1010 and has a thickness of about 2 mm to about 4 mm. The muscularis mucosa 1010 is the outer muscle layer of the bladder 1000 positioned between the lamina propria 1006 and the adipose layer 1012 and has a thickness of about 5 mm to about 8 mm. The adipose layer 1012 is a layer of fat surrounding the entire bladder 1000.

Intravesical instillation of any device or self-embedding particles need to attach themselves to the urothelium 1004. The urothelium 1004 can be thought of as a dynamic coating covering the entire bladder 1000. This layer is dynamic because its absorptive properties, due to high vascularity, play a central role in understanding intravesical delivery and the ability of the drug delivery device 102 to be efficacious in delivering a desired “load” of API(s) 404.

Any chemical instilled into the bladder 1000 has minimal absorption into the blood stream due to the tight junctions between urothelial cells in the urothelium 1004 that prevent high levels of systemic chemical absorption from occurring. Accordingly, drug delivery device 102 can be tailored for penetration of the urothelium 1004 and delivery of the API 404 for treatment of the bladder 1000. As discussed above, APIs 404 that benefit from local delivery can include, but are not limited to, any chemotherapy for various neoplasms. In further embodiments, the APIs 404 can be any class of drug that influence contractibility of the bladder and/or anti infectives.

The present disclosure has been described above by way of example only within the context of the overall disclosure provided herein. It will be appreciated that modifications within the spirit and scope of the claims may be made without departing from the overall scope of the present disclosure.

1. A drug delivery device, comprising:
an enteric capsule enclosing an internal volume;
a plurality of drug containing particles positioned within the internal volume, wherein each of the plurality of drug containing particles includes a matrix body and an active pharmaceutical ingredient (API) distributed within the matrix body;
wherein the plurality of drug containing particles are configured to penetrate tissue.

2. The drug delivery device of claim 1, wherein the enteric capsule comprises an outer layer overlying an inner layer.

3. The drug delivery device of claim 2, wherein the outer layer is soluble within the stomach and the inner layer is soluble within the small or large intestine.

4. The drug delivery device of claim 1, wherein the enteric capsule comprises a single outer layer.

5. The drug delivery device of claim 1, wherein the API is selected from one or more of peptides, antisense oligonucleotides greater than 500 Da, cytokines, monoclonal antibodies, chemotherapy drugs, PD-1 inhibitors, PD-L1 inhibitors, and combinations thereof.

6. The drug delivery device of claim 5, wherein at least one of:
the chemotherapy drugs are selected from one or more of Gemcitabine, Cisplatin, Carboplatin, Fluorouracil (5FU), and combinations thereof;
the PD-1 inhibitors are selected from one or more of Pembrolizumab, Nivolumab, Cemiplimab, and combinations thereof; or
the PD-L1 inhibitors are selected from one or more of Atezolizumab, Avelumab, Durvalumab, and combinations thereof.

7. (canceled)

8. (canceled)

9. The drug delivery device of claim 1, wherein the matrix body is formed from a biodegradable polymer.

10. The drug delivery device of claim 9, wherein the biodegradable polymer is selected from one or more of poly(lactic-co-glycolic acid) [PLGA] polymers, PLGA copolymers, poly(caprolactone)s (PCLs), poly(alkyl cyanoacrylates) (PACAs), poly(ortho esters), poly(anhydrides), poly(amides), poly(ester amides), poly(phosphoesters), microbial release polymers, and combinations thereof.

11. (canceled)

12. (canceled)

13. The drug delivery device of claim 1, wherein an aspect ratio of the plurality of drug containing particles is within the range from about 5 to about 100.

14. The drug delivery device of claim 1, wherein the plurality of drug containing particles have a shape including at least one vertex.

15. The drug delivery device of claim 1, wherein the plurality of drug containing particles have an elastic modulus within the range from about 1 GPa to about 10 GPa.

16. The drug delivery device of claim 1, wherein an axial failure force of the plurality of drug containing particles is within the range from about 1 N to about 10 N.

17. The drug delivery device of claim 1, wherein the plurality of drug containing particles have a sharpness within the range from about 0.1 μm to about 20 μm.

18. The drug delivery device of claim 1, wherein the surface of at least a portion of the drug containing particles is functionalized with a mucoadhesive.

19. A method of preparing a drug delivery composition, comprising:
forming a plurality of drug containing particles, wherein each of the plurality of drug containing particles includes a matrix body and an active pharmaceutical ingredient (API) distributed within the matrix body, wherein the drug containing particles are configured to penetrate tissue; and
enclosing the plurality of drug containing particles in an internal volume of an enteric capsule, wherein the enteric capsule is configured to release the plurality of drug containing particles from the cavity after placement within a gastrointestinal tract of a patient for a predetermined amount of time.

20. The method of claim 19, wherein the enteric capsule comprises an outer layer overlying an inner layer.

21. The method of claim 20, wherein the outer layer is soluble within the stomach and the inner layer is soluble within the small or large intestine.

22-36. (canceled)

37. The method of claim 19, wherein forming the plurality of drug containing particles comprises:
casting a liquid precursor of the drug containing particles;
solidifying the liquid precursor to form a sheet of the drug containing particles;
urging a portion of the sheet within cavities of a mold to form discrete drug containing particles; and
removing the discrete drug containing particles from the mold.

38. A method of orally delivering a drug to the gastrointestinal tract, comprising orally administering an effective amount of the drug delivery device of claim 1.

39. The method of claim 38, further comprising orally administering one or more second capsules that are substantially insoluble within the GI tract.

40. The method of claim 39, wherein the surface of the drug containing particles are functionalized with a compound configured to promote adsorption of the drug containing particles to the one or more second capsules.

41. The method of claim 39, wherein the one or more second capsules are configured to swell within the GI tract.

42. (canceled)

43. The method of claim 39, wherein the second capsules are configured for electrostatic affinity with the drug containing particles.
